(19) United States
(12) Patent Application Publication
(10) Pub. No.: US 2025/0241756 A1
(43) Pub. Date: Jul. 31, 2025

(54) Clips Implanted in the Heart and Great Vessels That Allow Their Complete or Partial Removal, Kits and Methods to Perform Such Removal

(71) Applicant: v2vmedtech, Inc., Maple Grove, MN (US)

(72) Inventors: Michael McDonald, Cordova, TN (US); Vinayak N. Bapat, Edina, MN (US)

(21) Appl. No.: 18/849,141

(22) PCT Filed: Mar. 27, 2023
(86) PCT No.: PCT/US2023/016409

§ 371 Date: Sep. 20, 2024

Related U.S. Application Data
(60) Provisional application No. 63/269,916, filed on Mar. 25, 2022.

(51) Int. Cl. A61F 2/24; A61B 17/00

(52) U.S. Cl. CPC A61F 2/2463; A61B 17/00234; A61B 2017/00243; A61B 2017/00305; A61B 2017/00358; A61B 2017/00862; A61B 2017/00929; A61B 2217/007; A61F 2220/0008

(57) ABSTRACT

A device implanted within the heart and great vessels, such as a valve clip, includes inbuilt features that can allow its removal, partial or complete, at a later date. The inbuilt features include one or more conductors configured to conduct energy. The energy is used to separate the clip or part of the clip from the surrounding tissue. A system or kit to facilitate this removal includes an inner catheter having a distal end configured to engage and couple with the clip, and a conductor configured to conduct energy between a source of energy and the distal end of the inner catheter. A method to facilitate this removal involves transferring the energy to the clip to cause detachment and separation of at least part of the clip from the surrounding tissue and withdrawing at least part of the clip into an outer catheter.

### FIELD AND BACKGROUND OF THE INVENTION

The invention relates generally to medical devices and methods. More particularly, the invention relates to devices that are implanted in the heart, especially clips with an integrated feature that can aid removal or modification of the clips at a future date and the systems, kits, and methods for doing so.

The mitral valve helps maintain a unidirectional flow between the left atrium and the left ventricle, and the tricuspid valve helps maintain a unidirectional flow from the right atrium to the right ventricle (FIG. 1A). Mitral Regurgitation (MR) is a commonly encountered valvular condition where the anterior and posterior leaflets of the mitral valve fail to close properly, resulting in backward flow from the left ventricle to the left atrium (FIG. 1B), while Tricuspid Regurgitation (TR), which is also a common condition, results when the three leaflets fail to coapt properly (FIG. 1C). The prevalence of these conditions increases with age.

Mitral regurgitation can result in numerous adverse consequences, such as heart failure due to left ventricular dysfunction, atrial fibrillation, pulmonary hypertension, and death. Published guidelines recommend surgical or transcatheter correction of mitral regurgitation to improve the clinical condition. Similarly, tricuspid regurgitation, where the blood regurgitates from the right ventricle to the right atrium, can cause devastating complications, such as liver failure, and has a very high surgical risk.

Transcatheter correction of mitral and tricuspid regurgitation by implantation of a valve clip (MitraClip™ system from Abbott Vascular and PASCAL from Edwards Lifesciences) has become a standard therapy for patients at high risk for open surgical corrective procedures. Such clip implantation procedures are performed through a guiding catheter that is inserted into the right femoral vein. One or more valve clips can be delivered through the guiding catheter and implanted to re-approximate the anterior and posterior mitral leaflets (often referred to as an “edge-to-edge” repair) (FIG. 2A) or between any two leaflets of the tricuspid valve (FIG. 2B, 2C). The MitraClip™ is a metallic implant made of a cobalt chromium alloy and covered with a fabric mesh, while the PASCAL device is made of Nitinol and fabric mesh.

To date, over 75,000 edge-to-edge procedures using these two devices have been performed globally, and there are currently nearly 1,500 procedures performed each month. The edge-to-edge procedure has been found to be a very safe procedure and provides a therapeutic option for patients at high surgical risk.

There are, however, some significant limitations to these two technologies. Not all patients achieve satisfactory MR reduction at the time of the procedure or clinical benefit, and up to 1 in 5 patients may have a return of significant MR or have a need for a repeat intervention within 6 months of the procedure. The only option currently available is removal, which typically requires open surgical excision. Fabric on the outside of the current clip results in excessive tissue growth, and hence it is not possible to remove the clip without cutting the leaflets around the clip.

Another option is the implantation of a transcatheter valve, but the presence of clips prevents implantation of these devices, and hence the removal of the clip/clips is an essential step prior to the implantation of these devices. If the valve clip can be removed without surgery, the option of transcatheter implantation of a heart valve can avoid a high-risk surgery in these patients. Such removal is being attempted by purpose-made devices which use wires to go around the clip and cut the leaflet tissue but cannot guarantee removal or result in excessive tissue damage resulting in hemodynamic instability.

It would therefore be desirable to design the clip with inbuilt features that can allow the application of energy to the clip through a transcatheter approach using a specially designed catheter and methods, which results in the detachment of the clip from one or all leaflets and for the transcatheter removal of clip/clips. It would be particularly desirable that such devices, systems, kits, and methods for the transcatheter removal of clips and other implanted prostheses from heart valves would leave the heart valve in condition to receive a subsequent transcatheter procedure, such as prosthetic valve implantation, to treat the valve pathology.

### SUMMARY

The disclosure describes preferred embodiments of clips for re-approximating leaflets of a cardiac valve. In these preferred embodiments, the clips comprise a post, a lower jaw, and an upper jaw. The post has a proximal end and a distal end. The lower jaw is coupled to the distal end of the post. The upper jaw is coupled to the post above the upper jaw. The lower jaw and the upper jaw are configured to catch and hold a leaflet of a cardiac valve. The post includes a first conductor (preferably a multiconductor) configured to conduct energy between the proximal end of the post and both the lower jaw and the upper jaw. The lower jaw includes a second conductor (optionally a multiconductor) configured to conduct the energy between the first conductor and a first exposed surface of the lower jaw that is designed to contact the leaflet, preferably directly. The upper jaw includes a third conductor (optionally a multiconductor) configured to conduct the energy between the first conductor and a first exposed surface of the upper jaw that is designed to contact the leaflet, preferably directly. In the preferred embodiments, portions of the post, as well as the lower and upper jaws, do not conduct the energy (i.e., are insulators). In other embodiments, some or all of these portions can be omitted.

The disclosure describes preferred embodiments of surgical kits for removing a clip implanted for re-approximating leaflets of a cardiac valve. In these preferred embodiments, the kits comprise a source of energy, an inner catheter, an outer catheter, and, optionally, one of the preferred embodiments of the clips described above. The inner catheter includes a shaft having a distal end configured to engage and couple with the clip. The inner catheter also includes a conductor (preferably a multiconductor) configured to conduct energy between the source of energy and the distal end of the shaft of the inner catheter. The outer catheter includes a shaft having a lumen sized the receive the inner catheter and at least part of the clip.

The disclosure describes preferred embodiments of methods for removing a clip implanted for re-approximating leaflets of a cardiac valve. Optionally, the clip to be removed may include one or more aspects of one of the preferred embodiments of the clips described above. In these preferred embodiments, the methods include the step of providing a surgical kit including a source of energy, an inner catheter including a shaft having a distal end and a conductor, and an outer catheter including a shaft having a lumen. The methods include the steps of inserting the inner catheter through the lumen of the outer catheter, and introducing the inner catheter in a heart chamber. The methods include the step of engaging and coupling the distal end of the shaft of the inner catheter with the clip. The methods include the steps of conducting energy between the source of energy and the distal end of the shaft of the inner catheter, and transferring the energy to the clip to cause detachment and separation of at least part of the clip from cardiac tissue. The methods include the step of withdrawing the at least part of the clip into the outer catheter. Optionally, the clip may include one or more aspects of one of the preferred embodiments of the clips described above.

Specific features of the preferred embodiments are recited in the claims. It is contemplated that various combinations or sub-combinations of the specific features may be described in the paragraphs of the detailed description and the drawings. Parallel combinations or sub-combinations without incorporating other aspects described in the same paragraph(s) or drawing(s) may also describe the invention. Furthermore, implementation details of the specific features are described in the paragraphs of the detailed description and the drawings. It is also contemplated that these implementation details may be used without incorporating other aspects described in the same paragraph(s) or drawing(s) to further qualify this specific feature of the invention or any combinations or sub-combinations of features of the invention involving this specific feature.

### BRIEF DESCRIPTION OF DRAWINGS

For a more detailed description of the embodiments of the present disclosure, reference will now be made to the accompanying drawings, wherein:

FIG. 1A illustrates the anatomy of the chambers of the human heart and illustrates the position of the mitral valve, tricuspid valve, and interatrial septum;

FIG. 1B illustrates the mitral valve anatomy with anterior and posterior leaflets;

FIG. 1C illustrates the tricuspid valve anatomy with three leaflets, anterior, posterior, and septal;

FIG. 2A illustrates a valve clip applied to the mitral valve leaflets, which results in a double orifice mitral valve opening;

FIG. 2B illustrates a valve clip between the anterior and the posterior leaflet of the tricuspid valve;

FIG. 2C illustrates a valve clip between the anterior and the septal leaflet of the tricuspid clip;

FIG. 3A illustrates a valve clip used to catch two leaflets, the valve clip having a feature for the removal of the valve clip, the feature including a conductor of energy located in a central position, the conductor being configured so that energy can be delivered to the margins, edge, or periphery of the jaws of the valve clip;

FIG. 3B illustrates a valve clip used to catch two leaflets, the valve clip having a feature for the detachment of the valve clip, the feature including a conductor that is configured to transfer energy to one leaflet resulting in the detachment rather than the removal of the valve clip;

FIG. 3C illustrates a valve clip used to catch two leaflets, the valve clip having a feature for the removal of the valve clip, the feature including a design variation of a conductor that conducts the energy;

FIGS. 4A-4B illustrate a lower jaw of a valve clip used to catch two leaflets with a feature for the removal of the valve clip, the feature including a conductor that can conduct energy on the margins, edge, or periphery of the clip jaws;

FIGS. 4C-4D illustrate a lower jaw of a clip used to catch two leaflets, the valve clip having a feature for the removal of the valve clip, the feature including a conductor, for example, a conductive coating, that can conduct energy to the entire clip jaw, and optionally with an insulating coating;

FIG. 5 illustrates a valve clip used to capture only one leaflet, the clip having a feature for the removal or detachment of the valve clip, the feature including a conductor of energy;

FIG. 6 illustrates an outer catheter that includes a handle, a side flush port, and a steerable distal end;

FIG. 7 illustrates an inner catheter that can be inserted through the outer catheter shown in FIG. 6 and includes a distal steerable tip and a clip-grasping feature configured to couple with a valve clip, wherein, through the core of the inner catheter, there is a mechanism to transfer energy from an energy source to the valve clip, wherein the mechanism includes cable(s) or wire(s);

FIG. 8A illustrates a coupling of the inner catheter shown in FIG. 7 with a valve clip from the atrial side after its insertion through the outer catheter shown in FIG. 6;

FIG. 8B illustrates the retraction of the inner catheter shown in FIG. 8A with the detached valve clip received into the outer catheter;

FIGS. 9A and 9B illustrate embodiments of clip-grasping features configured to couple with a valve clip;

FIGS. 10A-10C illustrate a valve clip used to catch two leaflets, the valve clip including a lower jaw made of an elongated loop of wire and an upper jaw that is sized to fit inside the elongated loop, the jaws being jaws for catching the valve leaflet tissue, the valve clip having a feature for the removal valve clip;

FIGS. 11A-11D illustrate the configuration of jaws shown in FIGS. 10A-10C; and

FIG. 12 illustrates jaws of a valve clip used to catch two leaflets, the clip having a feature for the removal of the valve clip, the feature including a conductor and ceramic coating.

As is customary, the drawings may not be drawn to scale for the sake of clarity.

### DETAILED DESCRIPTION

The disclosure generally describes a feature within valve clips such that their removal or modification is facilitated in the future. The valve clip has a feature (e.g., cables, wires, optic fibers, tubes, and bundles thereof) for removal or detachment, which can be activated by using an energy source, such as, but not limited to, electrical, radiofrequency energy of various kinds, or laser, to allow the removal or detachment of the valve clip from the leaflets, restoring the native valve orifice and allowing future repair or replacement therapy. In such procedures, for clip removal or detachment, the physician can insert specifically designed catheters either from the left atrium or left ventricle. A specially designed catheter can attach to the atrial end of the clip and activate the feature, which can detach the clip from the leaflets and allow its retrieval in the specially designed catheter.

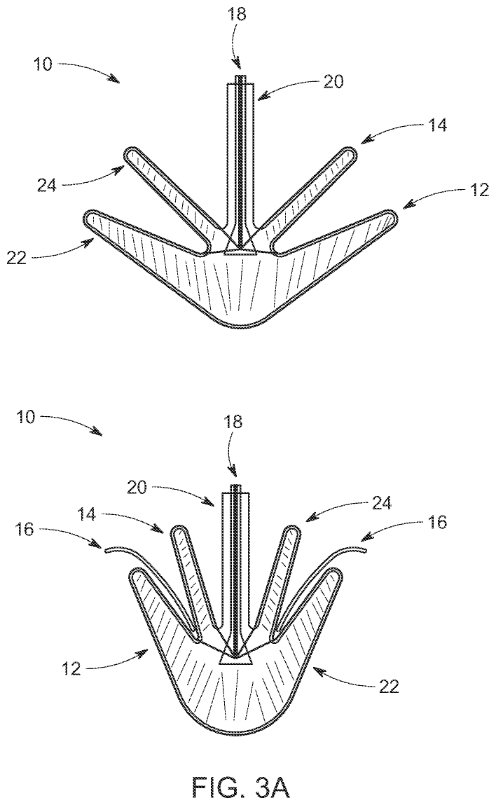

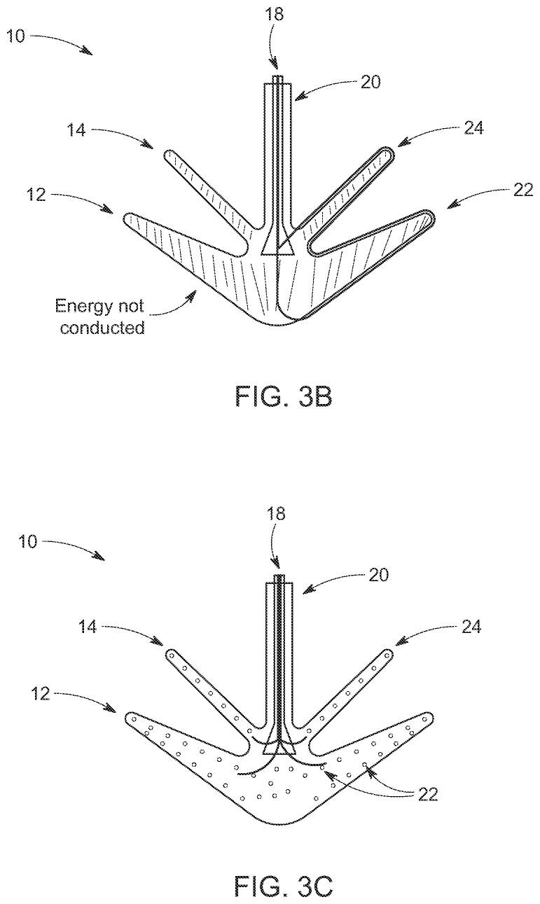

In a first aspect of the disclosure, preferred embodiments of the feature for the valve clip removal or detachment are such that the area of contact of the valve clip with the leaflet has the ability to conduct energy so that when activated, the energy can cut the tissue contact between the clip and the leaflets and can result in the detachment of the clip. The energy source, being localized to the contact area, can result in the least damage to the surrounding tissue. See, for example, the embodiments shown in FIGS. 3A and 3C.

The feature for the valve clip detachment can be configured so that energy can be passed to only one side of the clip, resulting in the detachment of the clip from one leaflet. See, for example, the embodiment shown in FIG. 3B. Energy can generally be passed to any subportion of the clip independently to other any subportions of the clip.

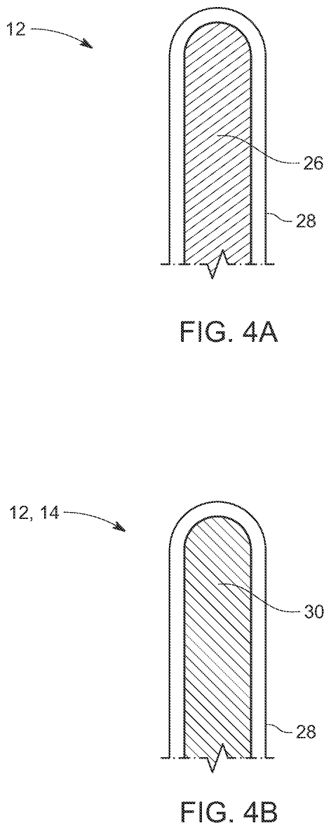

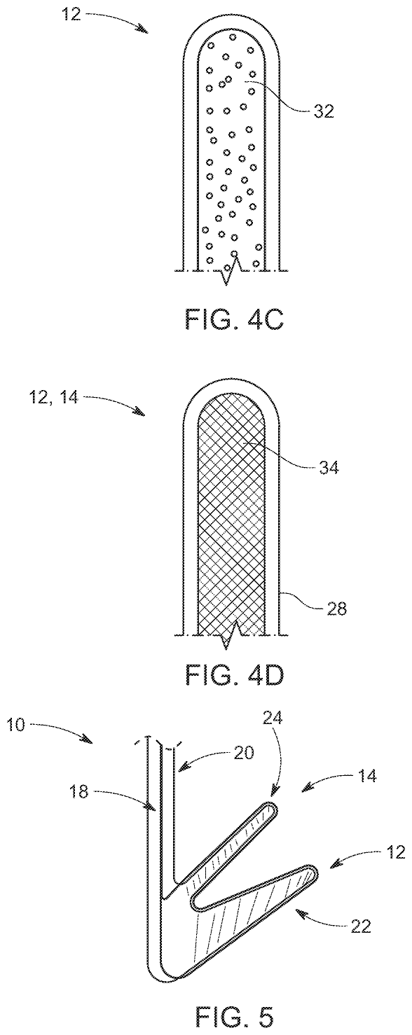

The feature for the valve clip removal or detachment can be configured so that energy can be conducted through the suitably placed metal elements, which are incorporated in the clip design and can be accessed through the atrial end of the clip. These metal elements are uninsulated and may be located at the margin, edges, or periphery of an essentially flat clip jaw (sometimes referred to as clip “paddles”), unlike the central portion of the clip jaw, which could be insulated. See, for example, the embodiments shown in FIGS. 4A and 4B. The central portion of any of the clip jaws can be made of fabric with or without a metal mesh, while the margins, edge, or periphery is made from a good conductor of energy. See, for example, the embodiment shown in FIG. 4A. The central portion of any of the clip jaws can be made of a polymer, optionally bioabsorbable, membrane with or without metal mesh, while the margins, edge, or periphery is again made from a good conductor of energy. See, for example, the embodiment shown in FIG. 4B.

The feature for the valve clip removal can include a structure in the clip that may be tungsten or other metals. The surfaces of the main or lower jaw (also referred to as the main or lower “paddle”) and the upper or gripper jaw (also referred to as the upper or gripper “paddle”) that come into contact with the valve leaflet tissue can be bare metal surfaces. See, for example, the embodiments shown in FIGS. 4C and 4D. For example, the feature for the valve clip removal can be configured so that energy can be conducted through the entire metal surface of the clip, and the conduction is facilitated by a special coating, such as, but not limited to, tungsten coating or tungsten construction of the body of clip jaws. For example, the central portion of any of the clip jaws can be further coated with tungsten. See, for example, the embodiment shown in FIG. 4C. Alternatively, the outer surfaces of the clip that can be exposed to the bloodstream can have a ceramic or another similar coating. The ceramic coating serves to insulate against electrical and thermal conductions. See, for example, the embodiment shown in FIGS. 4D and 12.

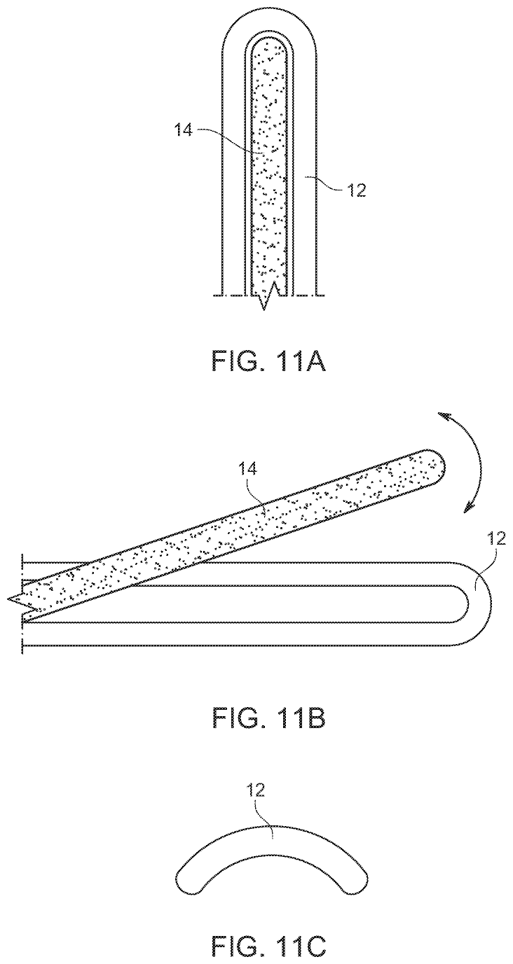

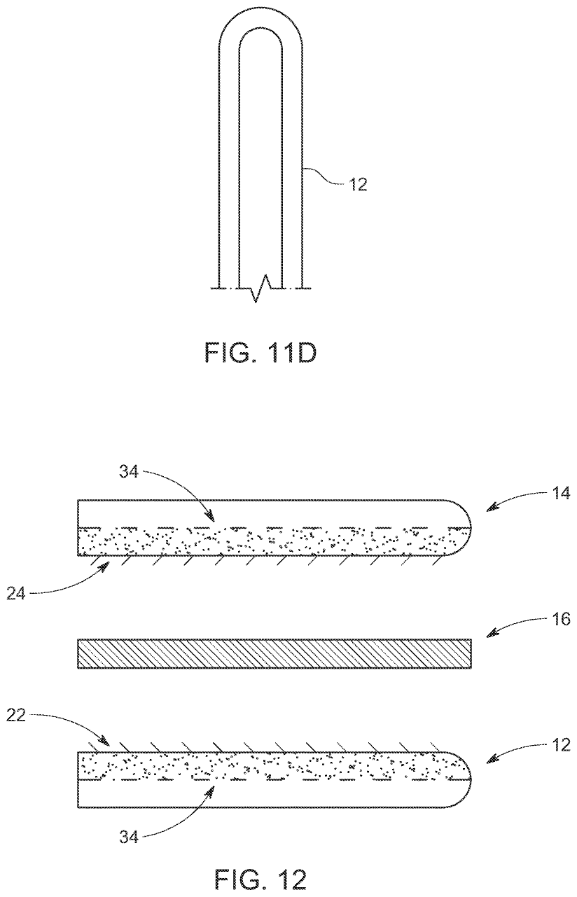

The surfaces of the clip jaws that contact tissue may have small protrusions for holding the valve leaflet tissue. The configuration of the lower or main jaw may be an elongated loop that the upper or gripper jaw can fit into when the jaws are in the folded position. See, for example, the embodiment shown in FIG. 11A-11D.

In a second aspect of the disclosure, preferred embodiments of a system or kit for excising an implanted clip comprise an outer catheter and an inner catheter.

The inner catheter that connects to the clip can be able to transmit electricity or other energy into the clip. This inner catheter can also have a flush port and channel. When the energy source is electrical, flush with D5W fluid (i.e., Dextrose 5% Water intravenous fluid) can be needed to isolate the electrical field.

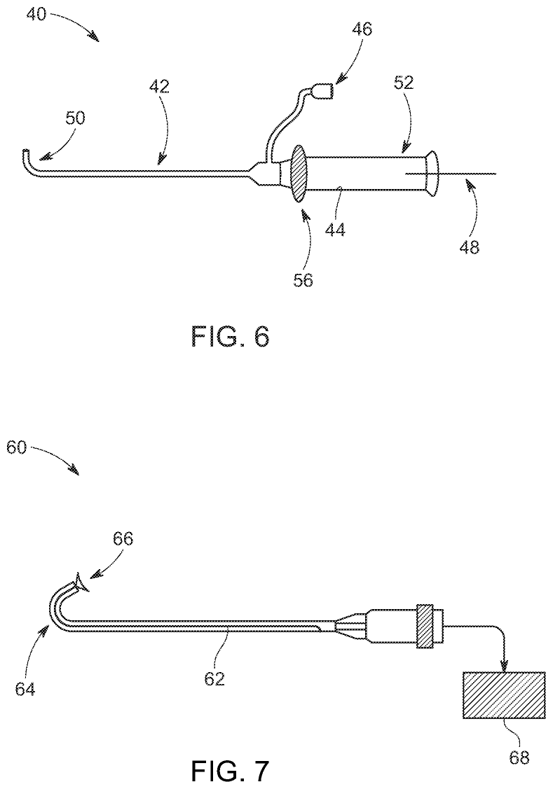

The outer catheter (as is shown, for example, in FIG. 6) is introduced into the right atrium and then into the left atrium when required. The outer catheter is configured to receive the inner catheter with a complete or partial clip.

The inner catheter (as is shown, for example, in FIG. 7) is configured to be introduced into the heart chamber adjacent to the valve leaflets on the atrial side of the clip for the application of the energy and grasping the clip.

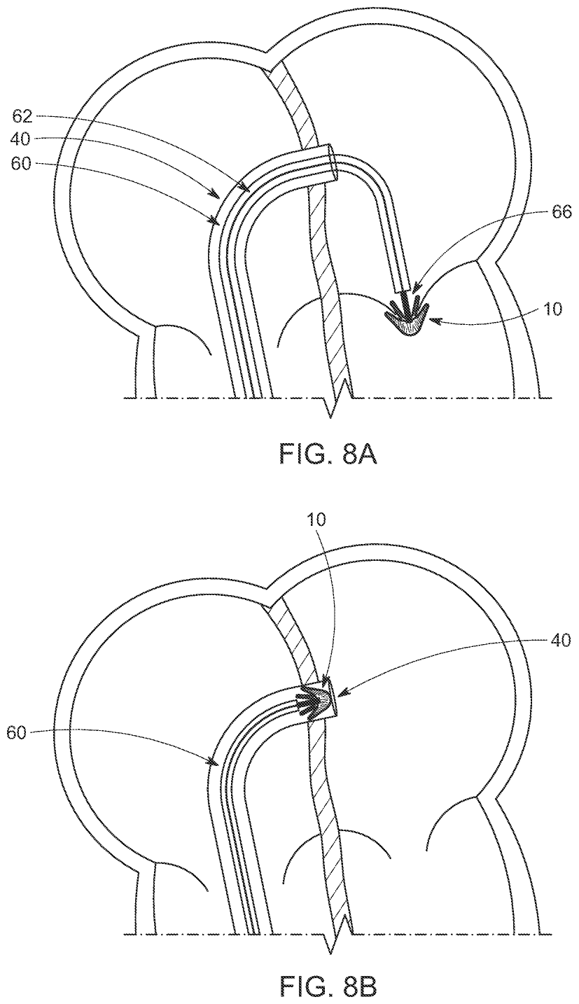

Both the inner and the outer catheters can have steering capability. The inner catheter can attach to the atrial or ventricular portion of the clip and can activate the feature that can allow separation of the clip or part of the clip from the tissue. Once separated, the inner catheter can be withdrawn into the outer catheter, and both catheters can be removed together with the retrieval of the clip. See, for example, the sequence shown in FIGS. 8A and 8B.

The outer catheter can remain in place and allow further procedure, be it valve replacement or balloon, or both.

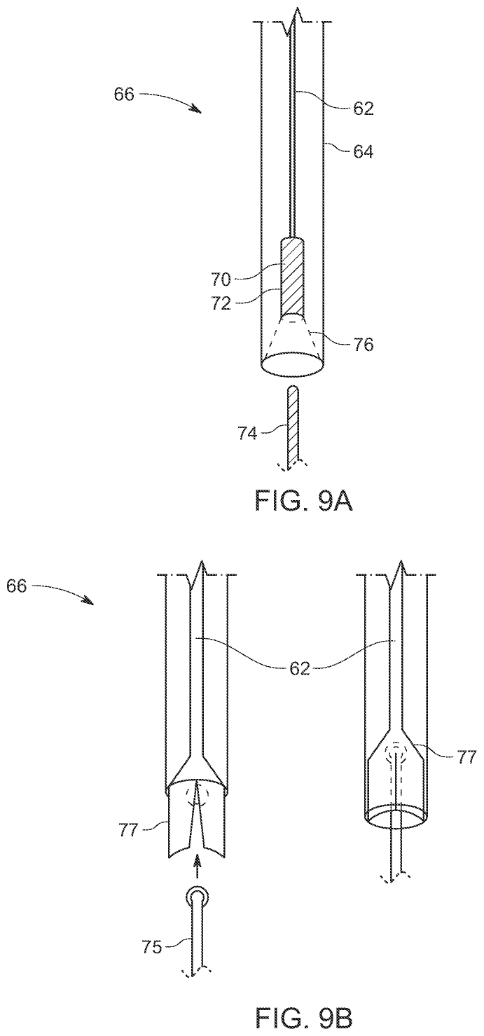

The inner catheter can typically have a distal end configured to engage and couple with the clip and include a mechanism to transfer energy from a source, as is shown, for example, in FIGS. 9A-9B. The shape of the valve clip can be typically easy to discern on fluoroscopy, which facilitates targeting and docking the inner with the clip during a removal procedure. Such docking has several advantages. First, docking can stabilize the valve clip relative to the catheter system, which is advantageous as the valves are moving during the beating heart procedure. Second, docking facilitates optimal activation and transfer of energy and limits it to a compact area. Third, once the clip is released from the tissue, it is held securely and withdrawn into the outer catheter, thus preventing embolization, as is shown, for example, in FIG. 8B.

The inner catheter, in addition, may have any one of a variety of clip-grasping features, such as a snare, biopsy-type forceps, or jaws that secure the valve clip to hold it after it has been freed from both leaflets so it can be removed from the body.

In a third aspect of the disclosure, preferred methods of removal of a clip previously implanted in the heart and great vessels are described. Typically, the clip includes a feature that can be integrated within the clip (i.e., an inbuilt feature), the feature being configured to facilitate the removal of the clip. These removal procedures are facilitated by systems, kits, or catheters described herein.

The valve clip can be located anywhere along the heart valve coaptation plane (central, medial, lateral, or commissural). In some embodiments, the valve clip can be removed from one of the coapting valve leaflets while being left attached in place to the other of the coapting valve leaflets. In other embodiments, the valve clip can be removed from all valve leaflets and extracted entirely from the heart.

The catheter that facilitates the removal procedure can be deployed from a left atrial aspect (transseptal or direct atrial access) or from the left ventricular aspect (transapical, direct ventricular puncture, or retrograde aortic access) when used for the mitral valve and right atrial or ventricular access in case of the tricuspid valve. The energy for removal can be applied to the valve clip by engaging the valve clip's inbuilt feature configured to facilitate removal with a clip-grasping feature provided with the catheter. Energy sources could be, but are not limited to, electrical, radiofrequency, or laser. By applying the energy, the removal, partial or complete, can result in the least damage to the tissue in contact with the clip, which can result in minimal instability in hemodynamics. The coupling of the catheter and the valve clip also prevents embolization and facilitates the removal of the clip from the body. The catheter may be used to remove valve clips from tricuspid valves as well as mitral valves. See, for example, the sequence shown in FIGS. 8A and 8B.

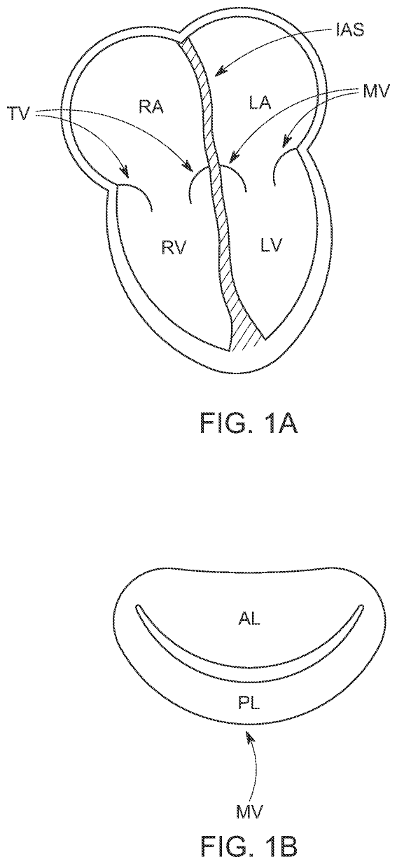

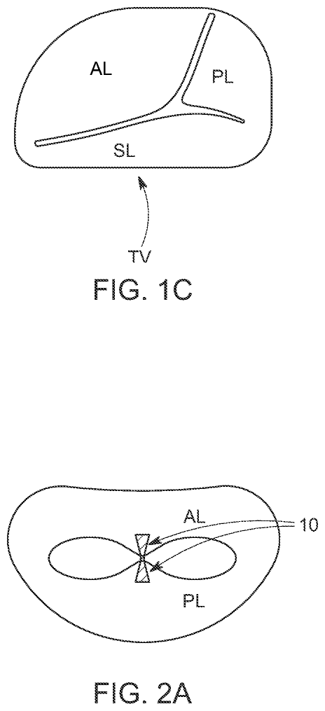

Referring to FIGS. 1A-1C, the mitral valve (MV) separates the left atrium (LA) from the left ventricle (LV), and the tricuspid valve (TV) separates the right atrium (RA) from the right ventricle (RV). For completeness, it is noted that the interatrial septum (IAS) separates the left chambers of the heart from the right chambers of the heart. Turning to FIG. 1B, mitral regurgitation results when the anterior leaflet (AL) and posterior leaflet (PL) of the mitral valve (MV) fail to close properly. Turning to FIG. 1C, tricuspid regurgitation results when the three leaflets (i.e., the anterior leaflet (AL), the posterior leaflet (PL), and the septal leaflet (SL) of the tricuspid valve (TV) fail to coapt properly.

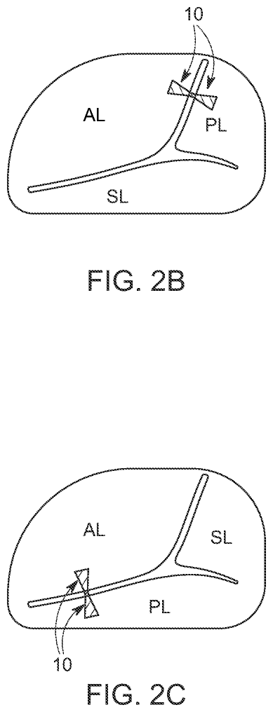

Valve clips may be implanted in the leaflets of a mitral valve (MV) as well as a tricuspid valve (TV) in a heart. As shown in more detail in FIG. 2A, the clip 10 in the mitral valve (MV) may be located near the middle of the valve opening between the anterior leaflet (AL) and the posterior leaflet (PL). The clip 10, however, could also be implanted at other locations between the leaflets away from the middle of the valve opening. As shown in more detail in FIGS. 2B, 2C, the clip 10 in the tricuspid valve may be located near between the anterior leaflet (AL) and the posterior leaflet (FL) or between any two leaflets. The clip 10, however, could also be implanted at other locations between the leaflets away from the middle of the valve opening.

Conducting energy to the contact surface of the jaws with the leaflets can result in the disengagement of the clip from the leaflet tissue and facilitate its removal. Referring to FIGS. 3A-3C, the clip 10 is designed such that the two jaws (i.e., bottom or lower jaw 12, and upper or gripper jaw 14) which catch the leaflet 16 have the ability to hold the leaflet 16 securely but at the same time allow conduction of the energy via conductor(s) 18 in the central portion of the post 20, conductor(s) 22 at the surface of the lower jaw 12, and conductor(s) 24 at the surface of the upper jaw 14. The jaws are preferably essentially flat, and can be referred to as “paddles.” The conduction by the conductors 22 and 24 could be limited to the paddle margins, edges, or periphery of the clip jaws 12 and 14, as is illustrated in FIG. 3A, or be spread and distributed over the entire surface of the clip jaws 12 and 14, as is illustrated in FIG. 3C. When desired, only one side of the clip may have conductive elements 22 and 24 to result in partial detachment from one leaflet only, as is illustrated in FIG. 3B.

Note that while the clip 10 is shown in the open configuration on the left of FIG. 3A, and in the closed configuration on the right of FIG. 3A, only the open configuration of the clip 10 is shown in FIGS. 3B and 3B. However, the clips of FIGS. 3B and 3C have closed configurations.

Referring to FIGS. 4A-4D, suitably placed metal elements, which are incorporated in the clip design (e.g., part of the conductors 22 and 24 shown in FIGS. 3A-3C), can be accessed by a source of energy through the atrial end of the clip (through the conductor(s) 18 shown in FIGS. 3A-3C). These metal elements are uninsulated and may be only at the periphery of the paddle surface of the clip jaws 12 and/or 14. For example, the metal elements may be implemented with a special coating, such as, but not limited to, tungsten coating 32 (as shown in FIG. 4C) or tungsten construction of the clip jaw.

Turning to FIGS. 4A, 4B, and 4D, the central portion of the clip jaw could be insulated (e.g., coated with insulating material or made of insulating material). For example, the central portion of any of the clip jaws can be made of fabric 26, with or without a metal mesh, while the margins, edge, or periphery 28 is made from a good conductor of energy. Alternatively, the central portion of any of the clip jaws can be made of a polymer, bioabsorbable membrane 30, with or without metal mesh, while the margins, edge, or periphery 28 is still made from a good conductor of energy. Alternatively, the central portion of any of the clip jaws can be covered by a ceramic coating 34, while the margins, edge, or periphery 28 is still made from a good conductor of energy.

Referring now to FIG. 5, a valve clip 10 that can be used to capture only one leaflet includes jaws 12 and 14 only on one side of the post 20.

Referring now to FIG. 6, an outer catheter 40 comprises a shaft 42 having a proximal end and a distal end. A control handle 44 is attached to the proximal end of the shaft 42, and a deflection knob is located in the handle 44. The outer catheter 40 can typically also include a flush port 46 and may have a central lumen 52 having one or more one-way valves adapted to receive a guidewire, dilator, or another instrument 48. As shown in FIG. 6, a distal region 50 of the outer catheter shaft 42 can be actively deflectable or “steerable” so that the region can be deflected by up to 180° and could be in all directions, as shown. Such deflection can be controlled by the deflection knob, and suitable deflection mechanisms 54 may include pull wires, slotted regions at the distal end of the shaft, or any other conventional catheter deflection technology. The flush port 46 can also allow infusion of fluid if required.

Referring now to FIG. 7, an inner catheter 60 comprises a shaft having a proximal end and a distal end. A control handle is attached to the proximal end of the shaft, and a deflection knob is in the handle. The inner catheter 60 may have a flush port and associated channel (not shown) and may have a central lumen adapted to receive a guidewire. A distal region 64 of the inner catheter shaft can be actively deflectable or “steerable” so that the region can be deflected by up to 180° and could be in all directions, as shown. Such deflection can be controlled by the deflection knob, and suitable deflection mechanisms may include pull wires, slotted regions at the distal end of the shaft, or any other conventional catheter deflection technology. The distal end of the inner catheter 60 includes a clip-grasping feature 66 (see also, for example, FIGS. 9A-9B) that can have the ability to engage and secure the atrial or ventricular end of the valve clip to apply energy to the valve clip feature for removal. The shaft of the inner catheter 60 also includes an inbuilt mechanism 62 (e.g., cables, wires, optic fibers, tubes, and bundles thereof) to transfer energy. A source of energy 68 can be connected to the inner catheter 60 at its proximal end. The attachment of the inner catheter to the external source of energy is illustrated, but the source of energy can be built-in within the catheter assembly.

As shown in FIG. 8A, the inner catheter 60 can attach to the atrial end of the clip 10 via the clip-grasping feature 66. Energy is conducted from the source via the inbuilt mechanism 62 and is transferred to the clip 10 at the and the clip-grasping feature 66. The energy can activate the feature of the clip 10 that can allow the separation of the clip or part of the clip from the tissue. Once the valve clip is detached from the leaflets, the inner catheter 60 with the valve clip attached to it can be withdrawn within the outer catheter 40 and removed from the body, as shown in FIG. 8B. Procedure steps can remain the same irrespective of the energy source used.

Referring to FIGS. 9A and 9B, the inner catheters used for clip removal can include clip-grasping features 66 adapted to the design of the clip to be removed. Examples of clip-grasping features 66 include a box (female) thread and cone, and a deformable receptacle configured to capture a ball when the ball is pressed against the receptacle. Turning to FIG. 9A, a clip includes a post having an atrial end, and the atrial end of the post includes a threaded pin 74. The clip-grasping feature 66 includes a box thread 72 having thread 70, and cone 76. The cone 76 is used to direct the threaded pin 72 toward box thread 72. The pin may be docketed by rotating the inner catheter can be rotated to thread the threaded pin 74 into the box thread 72. Once coupled, the inbuilt mechanism 62 is connected to the conductors (e.g., conductors 18, 22, and 24) of the clip. Similarly, turning to FIG. 9B, the atrial end of the post includes a ball 75. The clip-grasping feature 66 includes a deformable receptacle 77. The deformable receptacle 77 is expanded when it is outside of the inner catheter (as shown on the left). The deformable receptacle 77 is compressed around the ball 75 when it is inside the inner catheter. The clip may be docketed by pushing the inner catheter to press the deformable receptacle against the ball. The clip-grasping feature 66 may couple a plurality of its contact zones each connected to a corresponding conductor in the inner catheter with a corresponding one of a plurality of contact zones of the post, where each of the plurality of contact zones of the post is connected to a corresponding conductor in the post.

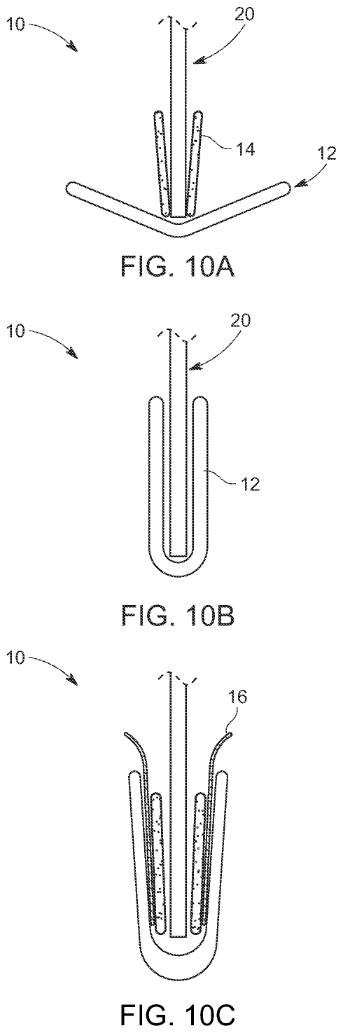

Referring to FIGS. 10A-10C, another embodiment of a valve clip 10, is illustrated. FIG. 10A shows the open configuration of the clip 10. FIG. 10B shows the closed configuration of the clip 10. FIG. 10C shows the closed configuration of the clip 10, the lower paddle 12 and the upper paddle 14 catch and hold a leaflet 16 of a cardiac valve. Similarly to previous embodiments, the valve clip 10 includes a lower or main paddle 12, an upper or gripper paddle 14, and a post 20. However, in this embodiment, the lower paddle 12 includes an elongated loop of wire. Also, the upper paddle 14 is sized to fit inside the elongated loop. The conductors in the post 20 are not illustrated in FIGS. 10A-10C, but are similar to the conductors in the clip posts shown in FIGS. 3A-3C.

Referring to FIGS. 11A-11D, the configuration of lower paddle 12 and upper paddle 14 shown in FIGS. 10A-10C is illustrated. FIG. 11A shows a side view of the paddles 12 and 14 in the closed position. FIG. 11B shows a frontal view of the paddles 12 and 14 in the open position. FIG. 11C shows a top view of the lower paddle 12 in the closed position. FIG. 11D shows a side view of the lower paddle 12. The upper paddle 14 can be inclined between 0 deg and 90 deg relative to the lower paddle 12. The tip of the lower paddle has a slight bow above the plane of the paddle.

Referring to FIG. 12, the configuration of the conductors is lower paddle 12 and upper paddle 14 shown in FIGS. 10A-10C is illustrated. In particular, the lower paddle 12 and the upper paddle 14 include conductive metal (e.g., tungsten) on the side designed to contact the valve leaflet 16 (e.g., be made or coated with conductive metal). Also, ceramic coating 34 may be used on the opposite sides to provide electric and thermal protection.

Although certain embodiments of the disclosure have been described in detail, certain variations and modifications can be apparent to those skilled in the art, including embodiments that do not provide all the features and benefits described herein. It can be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, can be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any method need not be performed sequentially.

What is claimed is:

1. A clip for re-approximating leaflets of a cardiac valve, comprising:
a post having an atrial end and a leaflet end; and
a lower jaw coupled to the leaflet end of the post and an upper jaw coupled to the post above the upper jaw, the lower jaw and the upper jaw being configured to catch and hold a leaflet of a cardiac valve;
wherein:
the post includes a first conductor configured to conduct energy between the atrial end of the post and both the lower jaw and the upper jaw;
the lower jaw includes a second conductor configured to conduct the energy between the first conductor and a first exposed surface of the lower jaw that is designed to contact the leaflet;
the upper jaw includes a third conductor configured to conduct the energy between the first conductor and a first exposed surface of the upper jaw that is designed to contact the leaflet.

2. The clip of claim 1,
further comprising another lower jaw coupled to the leaflet end of the post and another upper jaw coupled to the post above the upper jaw, the other lower jaw and the other upper jaw being configured to catch and hold another leaflet of the cardiac valve;
wherein the other lower jaw and the other upper jaw do not conduct the energy from the first conductor.

3. The clip of claim 1,
further comprising another lower jaw coupled to the leaflet end of the post and another upper jaw coupled to the post above the upper jaw, the other lower jaw and the other upper jaw being configured to catch and hold another leaflet of the cardiac valve;
wherein the other lower jaw and the other upper jaw are configured to conduct the energy from the first conductor.

4. The clip of claim 1, wherein
the lower jaw includes an energy insulator having a second exposed surface of the lower jaw that is designed to be exposed to blood; or
the upper jaw includes an energy insulator having a second exposed surface of the upper jaw that is designed to be exposed to blood.

5. The clip of claim 1, wherein
the lower jaw includes an energy insulator having a second exposed surface of the lower jaw that is designed to contact the leaflet, the second exposed surface of the lower jaw being located in a central portion of the lower jaw, and the first exposed surface of the lower jaw being located in a peripheral portion of the lower jaw; or
the upper jaw includes an energy insulator having a second exposed surface of the upper jaw that is designed to contact the leaflet, the second exposed surface of the upper jaw being located in a central portion of the upper jaw, and the first exposed surface of the upper jaw being located in a peripheral portion of the lower jaw.

6. The clip of claim 1, wherein the atrial end of the post includes a threaded pin or a ball.

7. The clip of claim 1, wherein the post includes an insulated portion surrounding a substantial portion of the first conductor.

8. The clip of claim 1, wherein:
the lower jaw includes an elongated loop of wire; and
the upper jaw is sized to fit inside the elongated loop.

9. A surgical kit for removing a clip implanted for re-approximating leaflets of a cardiac valve, comprising:
a source of energy;
an inner catheter including:
a shaft having a distal end configured to engage and couple with the clip; and
a conductor configured to conduct energy between the source of energy and the distal end of the shaft of the inner catheter; and
an outer catheter including:
a shaft having a lumen sized the receive the inner catheter and at least part of the clip.

10. The surgical kit of claim 9, wherein a distal region of the outer catheter shaft can be actively deflectable so that the distal region can be deflected by up to 180° in all directions.

11. The surgical kit of claim 9, wherein a distal region of the inner catheter shaft can be actively deflectable so that the distal region can be deflected by up to 180° in all directions.

12. The surgical kit of claim 9, wherein the conductor is located at the core of the shaft.

13. The surgical kit of claim 12, wherein the shaft of the inner catheter further includes a channel and a flush port.

14. The surgical kit of claim 9, wherein the shaft of the inner catheter includes a clip-grasping feature that is connected to the conductor and capable of transferring the energy to the clip.

15. The surgical kit of claim 14, wherein the clip-grasping feature includes at least one of a snare, biopsy-type forceps, jaws, a box thread and cone, and a deformable receptacle configured to capture a ball when the ball is pressed against the deformable receptacle.

16. A method for removing a clip implanted for re-approximating leaflets of a cardiac valve, comprising:
providing a surgical kit including a source of energy, an inner catheter including a shaft having a distal end and a conductor, and an outer catheter including a shaft having a lumen;
inserting the inner catheter through the lumen of the outer catheter;
introducing the inner catheter in a heart chamber;
engaging and coupling the distal end of the shaft of the inner catheter with the clip;
conducting energy between the source of energy and the distal end of the shaft of the inner catheter;
transferring the energy to the clip to cause detachment and separation of at least part of the clip from cardiac tissue; and
withdrawing the at least part of the clip into the outer catheter.

17. The method of claim 16, wherein:
the source of energy is electrical;
the shaft of the inner catheter further includes a channel and a flush port; and
the method further comprises flushing with Dextrose-based fluid using the channel and the flush port to provide electrical insulation.

18. The method of claim 16, wherein:
the shaft of the inner catheter includes a clip-grasping feature that is connected to the conductor; and
the method comprises transferring the energy to the clip through the clip-grasping feature.

19. The method of claim 18, wherein:
the clip includes a post having an atrial end and a leaflet end, a lower jaw coupled to the leaflet end of the post, and an upper jaw coupled to the post above the upper jaw, the lower jaw and the upper jaw being configured to catch and hold a leaflet of a cardiac valve;
the atrial end of the post includes a threaded pin;
the clip-grasping feature includes a box thread and cone; and
the method comprises rotating the inner catheter to couple the threaded pin to the box thread.

20. The method of claim 18, wherein:
the clip includes a post having an atrial end and a leaflet end, a lower jaw coupled to the leaflet end of the post, and an upper jaw coupled to the post above the upper jaw, the lower jaw and the upper jaw being configured to catch and hold a leaflet of a cardiac valve;
the atrial end of the post includes a ball;
the clip-grasping feature includes a deformable receptacle; and
the method comprises pushing the inner catheter to press the deformable receptacle against the ball.
